(19) United States
(12) Patent Application Publication
(10) Pub. No.: US 2020/0345858 A1
(43) Pub. Date: Nov. 5, 2020

(54) COMPOSITIONS AND METHODS FOR THE PREVENTION AND TREATMENT OF ERGOT ALKALOID TOXICITY

(71) Applicant: Linda Light, Suwanee, GA (US)

(72) Inventors: Linda Light, Suwanee, GA (US); David E. Nichols, Chapel Hill, NC (US)

(21) Appl. No.: 16/758,634

(22) PCT Filed: Oct. 22, 2018
(86) PCT No.: PCT/US18/56895

§ 371 Date: Apr. 23, 2020

Related U.S. Application Data
(60) Provisional application No. 62/575,749, filed on Oct. 23, 2017.

(51) Int. Cl. A61K 47/64; A61K 31/4985; A61K 31/48

(52) U.S. Cl. CPC A61K 47/643; A61K 47/64; A61K 31/48; A61K 31/4985; A61K 47/645

(57) ABSTRACT

Embodiments of the present invention provide materials and methods for preventing and treating ergot-based toxicity in animals, including humans. In particular, the present disclosure provides materials and methods for ameliorating the harmful physical manifestations of various diseases caused, at least in part, by ergot-based toxicity, including but not limited to caudal heel pain syndrome, idiopathic headshaking, pituitary pars intermedia dysfunction, metabolic syndrome and laminitis in horses; fescue foot, infertility and summer slump in cattle, sheep and goats; and neurologic, mental and somatic disorders in humans.

### CROSS-REFERENCE TO RELATED APPLICATIONS

The present application claims priority to U.S. Provisional Application No. 62/575,749, titled “COMPOSITIONS AND METHODS FOR THE PREVENTION AND TREATMENT OF ERGOT ALKALOID TOXICITY,” filed on Oct. 23, 2017, the entire disclosure of which being hereby expressly incorporated herein by reference.

### BACKGROUND

The USDA estimates that the agricultural industry loses one billion dollars per year due to diseases caused by ergot alkaloids, yet prevention and treatment so far have been only mildly successful. In addition to lack of success, attempted mitigation of ergot alkaloid toxicity has introduced more potential human exposure to drug residues in meat.

Ergot or ergot fungi refers to a group of endophytic Epichloe fungi and the ergot alkaloids produced by these non-yeast fungi that live inside grass plants, as well as ergot alkaloids produced by the Penicillium and Aspergillus species of fungi (that may not be endophytic) and bacteria of the genus Rhodococcus. Ergot alkaloids are found in cereal grains as well as all cold season forage grasses, wild rice, Bermuda grass, nut sedge and bahia grass.

The most well-known fungal producer of ergot alkaloids is Claviceps purpurea (“rye ergot fungus”). This fungus grows on rye and related plants, such as wheat and triticale, and produces alkaloids that can cause poisoning in humans and other mammals who consume grains contaminated with its fruiting structure (called ergot sclerotium). Claviceps includes about 50 known species. Economically significant species in the grain industry include, but are not limited to, C. purpurea (parasitic on grasses and cereals), C. fusiformis (on pearl millet, buffel grass), C. africana and C. sorghi (on sorghum). C. purpurea most commonly affects rye, triticale, wheat and barley.

The ergot sclerotium contains high concentrations (up to two percent of dry mass) of the alkaloid ergotamine, a complex molecule consisting of a tripeptide-derived cyclo-lactam ring connected via amide linkage to a lysergic acid (ergoline) moiety, and other alkaloids of the ergoline group such as ergocristine, ergocornine, ergocryptine, and lysergic acid amide that are biosynthesized by the fungus. Ergot alkaloids have a wide range of biological activities including effects on gastrointestinal health, circulation, neurotransmission and environmental perception.

In some cold season forage grasses, ergot alkaloids are found in great quantity without the presence of sclerotia, (e.g., in some members of the genus Lolium). The genus Lolium has a cosmopolitan distribution occurring on every continent except Antarctica. Fescue lameness has been reported across the United States as well as in New Zealand, Australia, and Italy. Tall fescue (Lolium arundinacium) is a cool-season perennial grass adapted to a wide range of soil and climatic conditions; it is used in Australia and New Zealand for stabilizing the banks of watercourses. It is the predominant pasture grass in the transition zone in the eastern and central USA.

The ubiquitous cold season perennial forage grass Lolium arundinacium causes two disease syndromes in cattle. The first resembles non-freezing cold injury in humans and is aptly termed “fescue foot” or “fescue lameness.” The second is epidemic hyperthermia commonly known as “summer slump.” Both of these disease syndromes cause loss of production in weight gain and negatively impact fertility and survival in cattle.

Fescue lameness due to consuming tall fescue in animals is caused by ergot alkaloids, especially ergovaline, produced by the endophytic fungus Neotyphodium coenophialum in tall fescue grass (Lolium arundinaceum, formerly Festuca arundinacea). It begins with lameness in one or both hind feet and may progress to necrosis of the distal part of the affected limb(s) leading to euthanasia. The tail and ears also may be affected independently of the lameness. In addition to gangrene of these extremities, animals may show loss of body mass, an arched back, and a rough coat, lower fertility rates, and preterm abortion.

The toxic substance ergovaline is comparable in its biologic action to ergotamine present in the sclerotia found in wheat, barley, oats and rye. Lysergic acid amide and lysergol are often found to be additionally present when ergovaline is found. The hyperthermic manifestation (also known as “summer slump”) of ergot poisoning is most prevalent in late summer when the seed heads of grass mature. The endophytic fungus N. coenophialum growing within the fescue plant can synthesize ergot alkaloids. Ergovaline has been detected in toxic fescue and constitutes ˜90% of the ergopeptide alkaloids produced. The ergovaline content of infected tall fescue often ranges from 100 to 1000 ppb, with >100 ppb producing symptoms of poisoning.

Ergovaline is an agonist for dopamine D2 receptors, serotonin 1a, 1b/1d, and 2a receptors along with α 1 and 2 adrenergic receptors, which initiates several physiologic abnormalities. Inhibition of prolactin secretion causes agalactia in horses and swine and reduced lactation in cattle. The dopaminergic effect also causes imbalances of progesterone and estrogen, associated with early parturition and spontaneous abortion for cattle. Mares experience prolonged gestation with weak, debilitated, and oversized fetuses. Ergot alkaloids may disturb the hypothalamic thermoregulatory center, leading to heat intolerance when environmental temperature exceeds 31° C. (88° F.). High temperatures increase the severity of epidemic hyperthermia or “summer slump,” in which a proportion of a herd of cattle exhibits symptoms of hyperthermia, reduced average daily weight gains and infertility, while low environmental temperature exacerbates the lesions of fescue lameness. This toxin appears to be a vasoconstrictor acting as an α₂-adrenergic agonist, as well as a serotonin 2a agonist on blood vessels; this promotes hyperthermia in hot weather and results in cold extremities during cold weather. Ergot alkaloid toxicity in cattle can also be caused by ergot alkaloids found in cereal grains, as well as straw made from cereal grain chaff and may be concentrated in distiller's dried grains.

Despite this information, the relationship between the presence of ergot alkaloids in the environments of other animals such as horses and the subsequent development of diseases like caudal heel pain syndrome, idiopathic headshaking syndrome, laminitis, and diseases involving dopamine and the D2 receptor in the hypothalamus such as Pituitary Pars Intermedia Dysfunction have not yet been elucidated.

Navicular disease in horses, now more aptly termed “caudal heel pain syndrome,” is a debilitating lameness. Lameness from all sources has an estimated loss to the equine industry of 80 million dollars per 160,000 horses. There are 9 million affected animals in the United States alone. Of this lameness, 47 percent is due to hoof problems according to surveyed owners of horses. Caudal heel pain syndrome occurs in the front feet of horses and can be the end of a horse's athletic career. It is one of the most common causes of lameness in the athletic horse. Caudal heel pain syndrome has been associated with a “toe first” landing, thin soles, poor digital cushion development, poor circulation in the digital cushion, and standing in a “goat on a rock” position at rest that keeps weight off the heels of the front feet. Manifestations of caudal heel pain syndrome can include lesions of the navicular bone, increased remodeling of the navicular (distal sesamoid) bone, increased connective tissue in the synovium of the navicular bursa and nutrient foramina, and arteriogram/venogram changes. Pedal osteitis (resorption of calcium from the solar margin of the third phalanx (P3)) is also a common manifestation of toe first landing and navicular disease. It had been demonstrated that the pathologic toe first landing creates the changes seen radiographically in the navicular bone, as well as the lesions in the deep digital flexor tendon frequently seen histopathologically.

It is generally understood that horses have an orthopedic hydraulic force dissipation system comprised of the digital cushion, lateral cartilages, and the complex mass of vessels that surround and run through the heel with numerous venous anastomoses. This hydraulic system maintains a negative pressure in healthy hooves deep within the digital cushion both at rest and in motion. The blood vessels in the heel, both arterial and venous, have smooth muscle around small vessels—much smaller than any other vessels with smooth muscle found within the vasculature of the horse's body. When the horse lands on its heel, these vessels control the outward flow of blood, thereby allowing for very accurate dissipation of force. It has been demonstrated that horses suffering from navicular disease do not have tachykinin receptors (nk-1) present on the small vasculature within this region of the hoof. Nk-1 receptors could not be detected using receptor autoradiography, yet a control group had detectable tachykinin receptors. It is thought that the neural controls of the microvasculature, which supply blood and venous drainage to and from the navicular bone and the distal phalanx, have been compromised, and the microvasculature has been destroyed in horses suffering from navicular disease, thus indicating insufficient arterial blood supply to these areas. It has also been observed that there is navicular bone erosion within the formation of synovial fossae and/or areas of both degenerative and regenerative bone within the navicular bone itself. In studies of horses with navicular disease, subchondral plate thickness, trabecular bone thickness, proteoglycan producing cells, percentage of bone within the distal phalanx, and bone area of the navicular bone were all significantly less than the average for all age group horses. The etiology of a horse's toe-first landing and the associated development of caudal heel pain syndrome are not yet known.

Idiopathic Headshaking Syndrome is comprised of a cluster of symptoms, also with no known etiology. Symptoms include vertical, sometimes violent, involuntary head movement exacerbated by wind, rain, sunlight, and/or exercise, accompanied by a desire of the horse to rub the nose aggressively on any object or the ground. Headshaking syndrome is unpredictable, involuntary, and can become dangerous for the rider, and it frequently marks the end of the horse's career. Euthanasia is elected in some cases. It has been compared to trigeminal neuralgia in humans. It is generally understood that the trigeminal nerve is involved in head shaking syndrome because using a lidocaine block close to the trigeminal entry into the skull ameliorates head shaking. Histologically, the trigeminal nerve appears to be normal, so demyelination disease or other nerve pathology does not account for the symptoms. In horses and humans, surgical ways to put more distance and/or padding between the vessel and nerve have had some success. In many cases, headshaking occurs seasonally (e.g., May-November).

Laminitis is a common and debilitating disease, which initially presents as an acutely painful condition of the feet that often warrants euthanasia. Frequency of laminitis across populations ranges from 1.5-34 percent. In a survey done by Purdue University, laminitis was responsible for 7.2 percent of horse deaths reported. The condition has multiple suspected etiologies. Acute pasture-associated laminitis is most frequently encountered and is often recurrent. The incidence and impact of laminitis led to the identification of derangements of carbohydrate and lipid metabolism and generalized or regional obesity as key risk factors for the disease. The conflation of obesity, hyperinsulinemia, and a susceptibility to laminitis are common associations, and cases that present with these signs are considered to possess the Equine Metabolic Syndrome (EMS) phenotype, frequently coinciding with Pituitary Pars Intermedia Dysfunction (PPID), although in the combination of metabolic syndrome with PPID, horses are often also overtly hyperglycemic. Despite the fact that the pathophysiologies of laminitis, obesity, and insulin regulation have been linked, not all laminitic horses or ponies are obese and/or insulin resistant.

Soft tissue inflammation in the laminae that connects P3 to the hoof wall causes pain and predisposes the patient to a separation of hoof wall and bone and, in severe cases, the bone penetrates the sole of the hoof. This disease has been associated with carbohydrate overload, equine metabolic syndrome, black walnut poisoning, equine Cushing's disease (PPID), excessive weight bearing (including road founder), and corticosteroid administration. More recent research refutes EMS and PPID as causes of laminitis.

The present invention provides a vaccine for the prevention of ergot-related diseases in animals, including mammals, such as cattle and humans. Vaccination with one or more ergot derivatives may provide protection to animals consuming ergot and its derivatives. Humans consuming cereal grains and products from animals that have fed on ergot-contaminated grains and grasses may also benefit from vaccination.

### SUMMARY

Embodiments contained in the present disclosure provide materials and methods for vaccines to prevent ergot toxicity in animals, including mammals, such as humans. A vaccine of the present invention comprises one or more ergot alkaloids connected to a carrier molecule, which is often but not always a peptide or protein. See, e.g., Gerdts et al., Vaccine, 2013, 31(4), 596-602. Said molecule or molecules generated are formulated as a vaccine as is known in the art and then provided to the animal via an appropriate route, which will often be injection.

Other features and advantages of the disclosure will be apparent from the following detailed description, and from the claims.

### DETAILED DESCRIPTION

Embodiments of the present invention provide materials and methods for treating ergot-based toxicity in animals. In particular, the present disclosure provides materials and methods for ameliorating the harmful physical manifestations of various diseases due to ergot-based toxicity, at least in part. Important diseases to prevent include summer slump and fescue foot in cattle along with caudal heel pain syndrome, idiopathic headshaking, and laminitis in horses.

The present disclosure addresses the need for therapeutic methods and treatments to reduce the harmful effects of ergot-based toxicity. For example, in some embodiments, the methods and treatments of the present disclosure can mitigate physical manifestations of ergot toxicity, such as preventing vasoconstriction in the extremities, reducing infertility and increasing average daily gains of animals. In some embodiments, preventing vasoconstriction will ameliorate one or more symptoms associated with diseases like caudal heel pain syndrome, idiopathic headshaking syndrome, and laminitis, for example.

Many animal species may be impacted by the presence of ergot derivatives in their diet, including, but not limited to mammals, such as horses, cows, pigs, sheep, goats, dogs, cats, humans and so on. While vaccination against ergot derivatives has been attempted (See, e.g., Fillipov et al., J. Anim. Sci., 1998, 76(9), 2456-2463; those authors found a “lack of long-lasting protection”), there remains a profound need for an answer to the debilitating impact of ergot derivatives in animals. The present invention provides vaccines based on a unique chemical design.

Embodiments of the present invention are included to demonstrate certain embodiments presented herein. It should be appreciated by those of skill in the art that the techniques disclosed in the examples that follow represent techniques discovered to function well in the practices disclosed herein.

This disclosure provides various immunogenic compounds and compositions described below. In general, many different forms of vaccine delivery are available to one of ordinary skill in the art. Descriptions of such methods are widely available in the art. One article which reviews such methods is Saroja, P. K., et al., Int. J Pharm. Invest., 2011, 1.2, 64-74. Additional discussion about vaccine delivery for animals may be found at Sharma S, Hinds LA. Formulation and delivery of vaccines: Ongoing challenges for animal management. Journal of Pharmacy & Bioallied Sciences 2012; 4(4), 258-266. The compounds of the invention are suitable for oral or mucosal delivery. However, the fructofuranosides or fructofuranosyl fructofuranosides may be more difficult to administer orally due to acid lability.

The compounds of the invention may be used as desired to vaccinate against any number of ergot-based toxicities. The user may choose which of the compounds are desired to be vaccinated against. It is not required that all compounds of the invention be used, only those for which protection is desired. The compounds of the invention may be in a sustained release preparation. These preparations may be utilized as a prophylactic to protect animals, such as mammals (e.g., humans, horses, etc.) from the toxic effects of ergopeptine and clavine alkaloids. Alternatively, the various preparations can be used as can be used as a therapeutic. As can be appreciated by one skilled in the art, there are many suitable ways to incorporate the immunogenic compounds described below into various embodiments of sustained release preparations (e.g., microcapsules, microsphere polymers, liposomes, polylactic acid preparations, etc.). Various embodiments disclosed herein may utilize the various immunogenic compounds described below in a biocompatible, biodegradable microsphere polymer or copolymer of polylactide or polyglycolide.

In various embodiments, the antigen can be incorporated, for example, into biodegradable microspheres, to produce an immunizing agent that will result in prolonged release of the antigen and therefore induce a long term immune response. Exemplary agents to make vaccine-containing microspheres include polyesters of polylactic acid, polyglycolic acid, their respective co-polymers, and combinations thereof. Exemplary microspheres may be produced using mild conditions that do not degrade or damage the various antigens. The antigens may be enclosed in the biodegradable matrix. Three exemplary methods used to produce these microspheres include phase separation (e.g., where drugs and polymers are dispersed or dissolved in a solvent and then the microspheres may be precipitated out by addition of silicon oil), solvent extraction (e.g., where drugs and polymers in solution are added to an aqueous solution of poly-vinyl alcohol to produce an oil-in-water emulsion and then the solvent is eliminated by adding water and the microspheres dried), and spray drying (e.g., where drugs and polymers are dissolved in a solvent and then spray dried). In any of the aforementioned procedures, after the spheres are formed, they may be dried and then separated into various sizes, for example, by sieving.

Factors which affect antigen release include erosion and breakdown of the particles, diffusion of the drug out of the matrix, solubility of the antigen, antigen molecular weight, antigen loading of the spheres and polymer molecular weight. For a given antigen the release rate is related to particle size; small particles release the antigen sooner than large particles. For prolonged release and immunization a mixture of small and large particles appears to be desirable as would be appreciated by an ordinary skilled artisan having the benefit of this disclosure.

The immunogenic compounds, described in further detail below, whether or not contained in a biodegradable microsphere, may also be placed in a pharmaceutically acceptable carrier, including but not limited to buffered saline or distilled water. Likewise, the immunogenic compounds can be mixed with a suitable adjuvant.

Chemical synthesis of the ergot derivatives can be achieved via methods known in the art. See, e.g., Recent Synthetic Studies on the Ergot Alkaloids and Related Compounds. The Alkaloids: Chemistry and Biology, Academic Press: San Diego, Calif., 2000; Vol. 54, pp 191-257. See also Liu and Jia, Nat. Prod. Rep., 2017, 34, 411-432.

In the aforementioned disclosed compounds, the carrier molecule may include, but is not limited to, a peptide or a protein. Exemplary proteins include a suitable immunogenic protein, which may include human serum albumin, bovine serum albumin, chicken globulin, ovalbumin, keyhole limpet hemocyanin, tetanus toxoid, polyarginine, polyhistidine, polytyrosine, polyserine, polyaspartate, and polylysine. A review of such molecules can be found at Pichichero, Michael E. Hum. Vaccin. Immunother., 2013, 9(12), 2505-2523. Methods for attaching such molecules are also well-known in the art.

In general, methods for synthesis of the subject compounds take advantage of the ability to deprotonate the indole NH using strong bases such as NaH in a suitable solvent such as dioxane, DMSO, and other solvents known to those skilled in the art. The resulting indolic anion is then treated with, for example, methyl-4-bromobutyrate to generate the 1-(4-carbomethoxypropyl)indole derivative. This methyl ester is then selectively hydrolyzed by mild base or lithium iodide to afford the free carboxylic acid. The resulting 1-(3-carboxypropyl)indole derivative is then coupled to the carrier molecule, usually a protein of interest, using standard peptide coupling reagents such as carbonyl diimidazole, a carbodiimide reagent, or the like. Following coupling the protein thus modified is purified to remove any excess uncoupled ergot derivative and byproducts of the coupling reagent, as shown below.

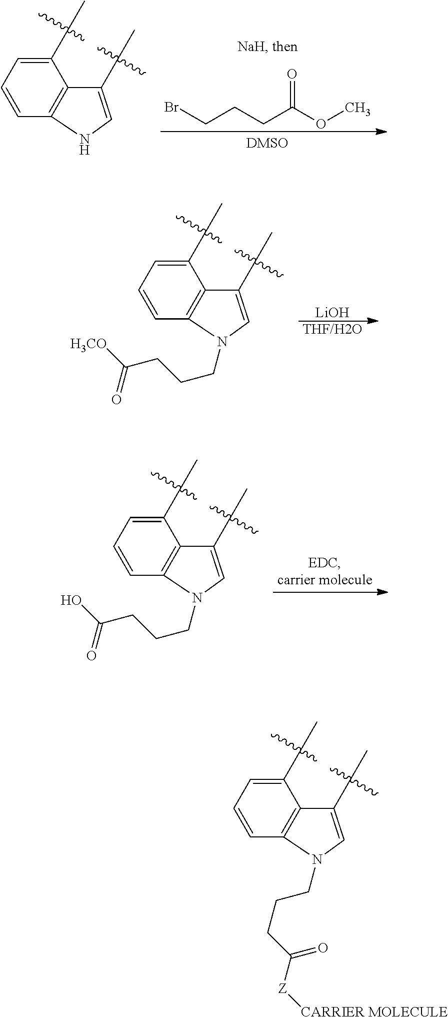

The ergot alkaloid derivatives themselves can be prepared from commercially available lysergic acid using known methods (U.S. Pat. No. 3,336,311 and Liu, H. et al., Org. Lett., 2017, 19(12), 3323-3326), while the clavines can also be prepared in accordance with known methods (see: Ergot Alkaloids. The Alkaloids: Chemistry and Pharmacology, Academic Press: San Diego, Calif., 1990; Vol. 38, pp 1-156; Liu, H. et al., Org. Lett., 2017, 19(12), 3323-3326; Oppolzer et al., Tetrahedron, 1983, 39(22), 3695-3705; McCabe, S. and Wipf, P., Org. and Biomol. Chem., 2016, 14, 5894-5913; McCamley, K. et al., J. Org. Chem., 2003, 68(25), 9847-9850; Schkeryantz J., et al., J. Am. Chem. Soc., 1999, 121, 11964-11975; Křen, V. et al., Appl. Microbiol. Biotech., 1990, 32, 645-650, Peng, Y. and Li, W.-D. Synlett, 2006, 1165-1168, Liu, Z., et al. J. Org. Chem., 2014, 79, 11792-11796).

In general, those molecules with free hydroxyl groups must be protected prior to the addition of methyl-4-bromobutyrate. An example is shown below.

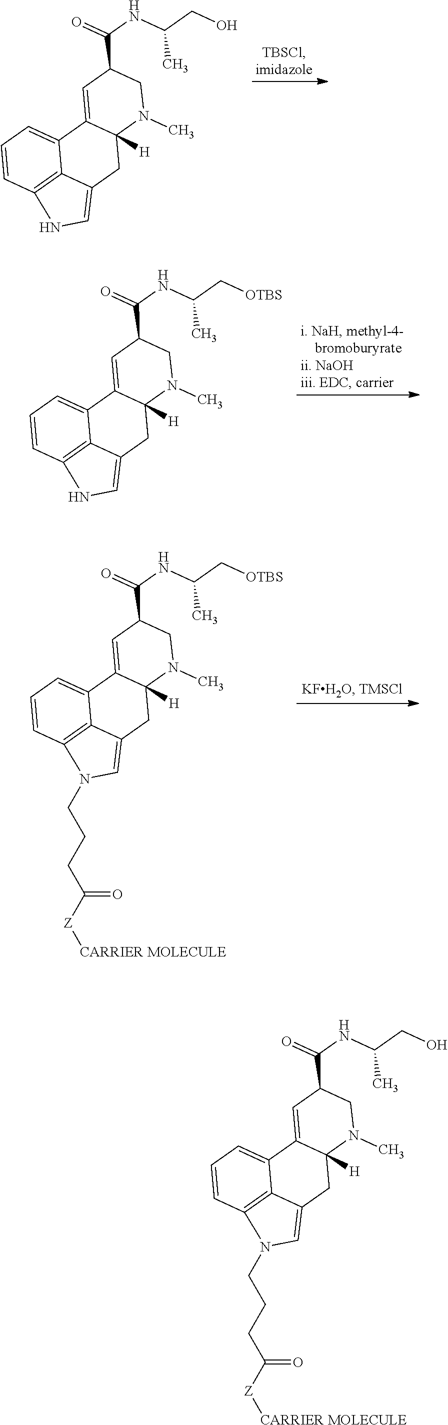

In situations in which a free amine is present, protection of the amine would also be required. A further example is shown below.

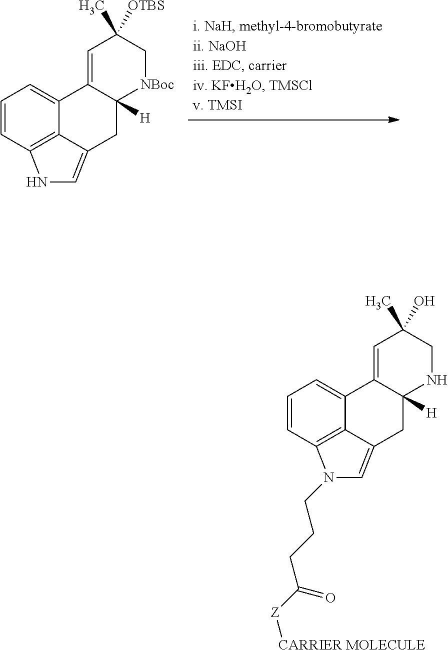

Finally, in those cases in which fructofuranosides or fructofuranosyl fructofuranosides are desired, the following reaction sequence may be employed to synthesize the compounds, as shown with the exemplary compound below.

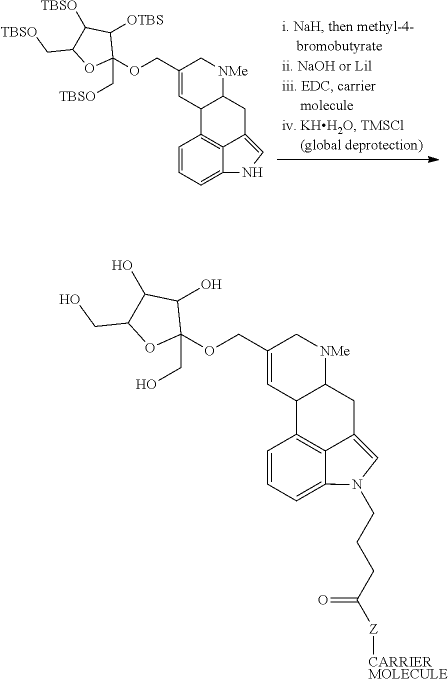

A composition for treating ergot-based toxicity in a subject is a compound of Formula 1:

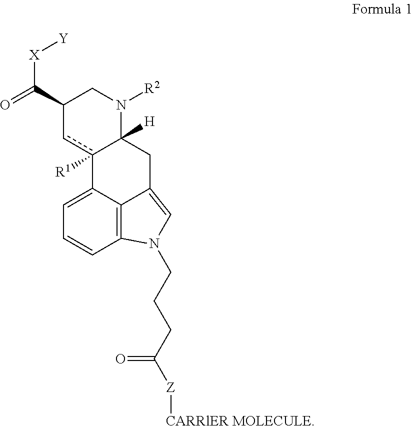

R¹ is selected from the group consisting of hydrogen and null in the case of the indicated double bond. Thus, the composition of Formula 1 includes one or more of the following structures:

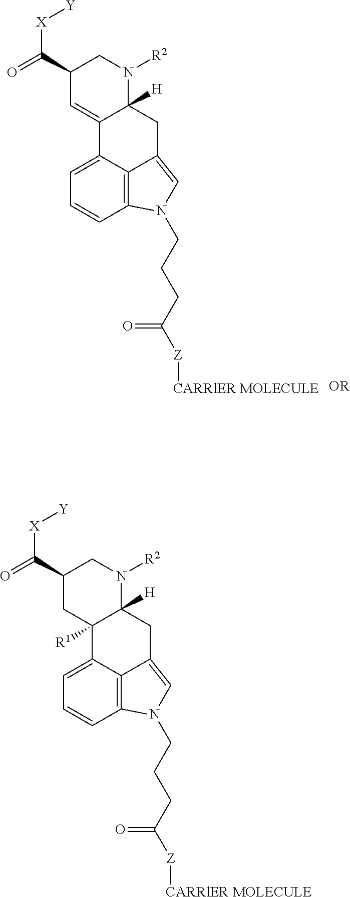

wherein R² is selected from the group consisting of methyl and hydrogen;

Z is selected from the group consisting of oxygen and nitrogen;

Y is selected from the group consisting of hydrogen, methyl, ethyl,

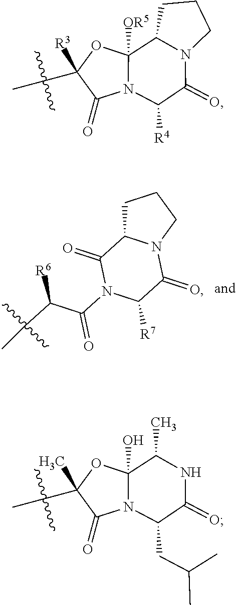

R³ is selected from the group consisting of methyl, ethyl, n-propyl, isopropyl, and sec-butyl;
R⁴ is selected from the group consisting of benzyl, ethyl, isopropyl, isobutyl, sec-butyl, n-butyl, 2-methyl-n-butyl, 2-methyl-n-propyl, and ethyl(methyl)sulfane;
R⁵ is selected from the group consisting of hydrogen and methoxy;
R⁶ is selected from the group consisting of isopropyl and sec-butyl;
R⁷ is selected from the group consisting of benzyl, ethyl, isopropyl, isobutyl, and sec-butyl; and

X is selected from the group consisting of a bond, carbon, nitrogen, oxygen, an amine (e.g., a primary amine, a secondary amine, or a tertiary amine), an amide (e.g., a primary amide, a secondary amide, or a tertiary amide), an ester, and an ether.

Exemplary compounds of Formula 1 include:

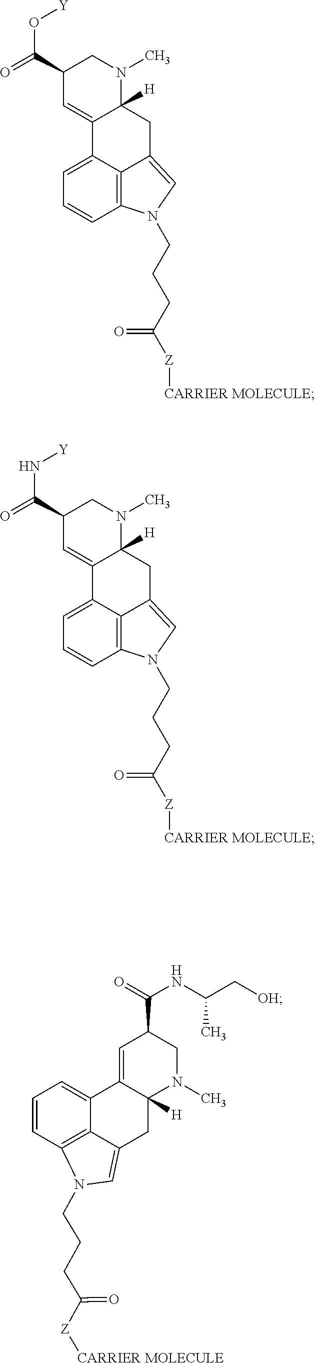

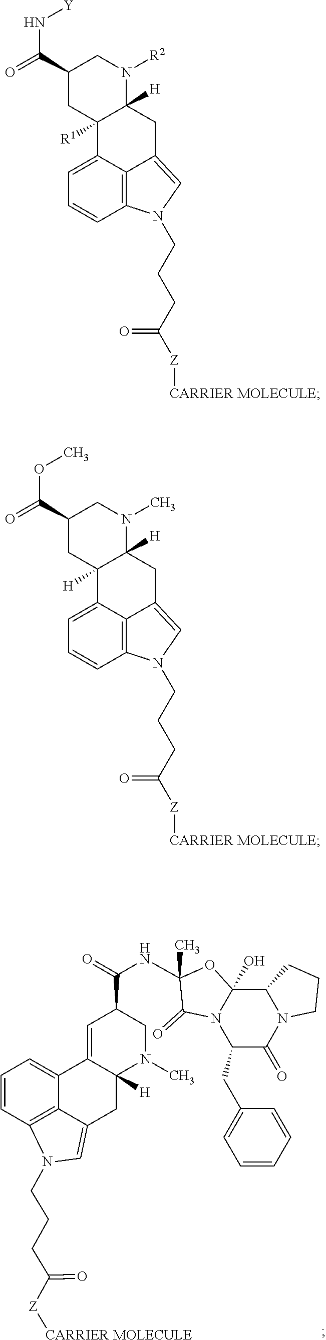

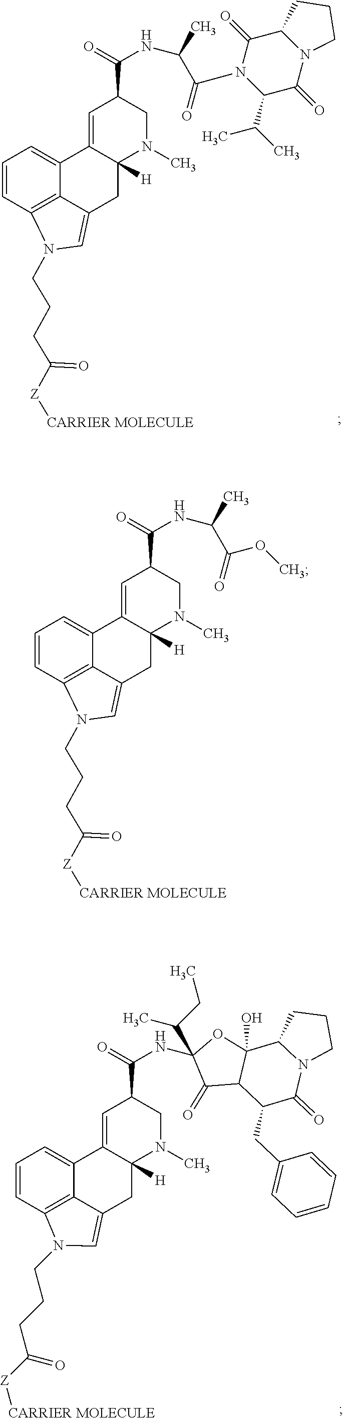

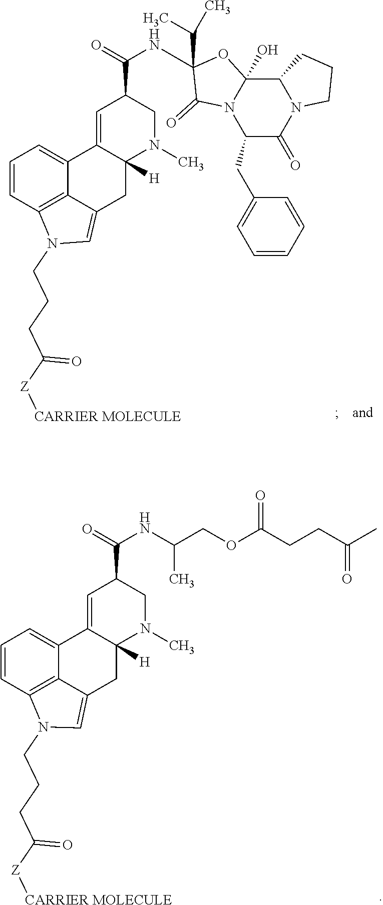

Also disclosed herein are compositions for treating ergot-based toxicity in a subject where the composition includes a clavine bonded to a carrier molecule. Exemplary clavines include the following compounds:

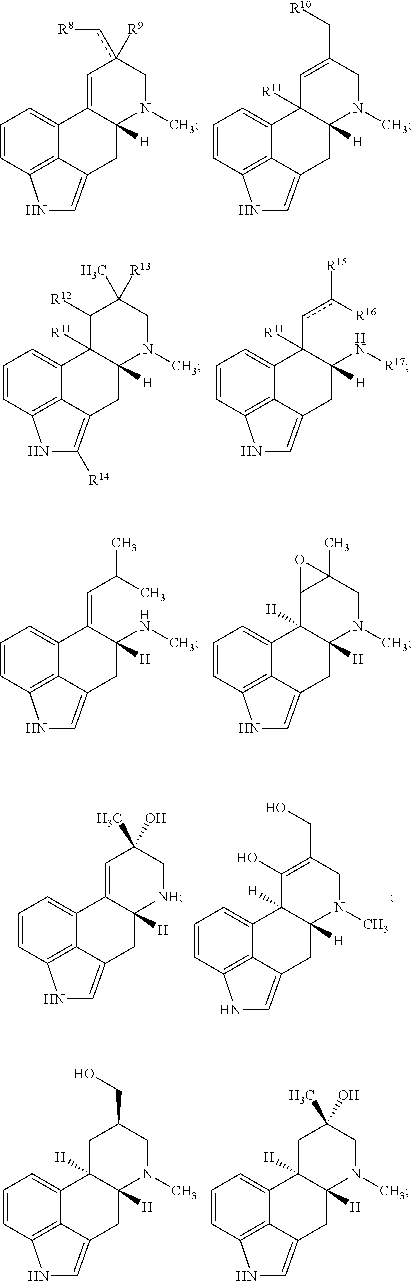

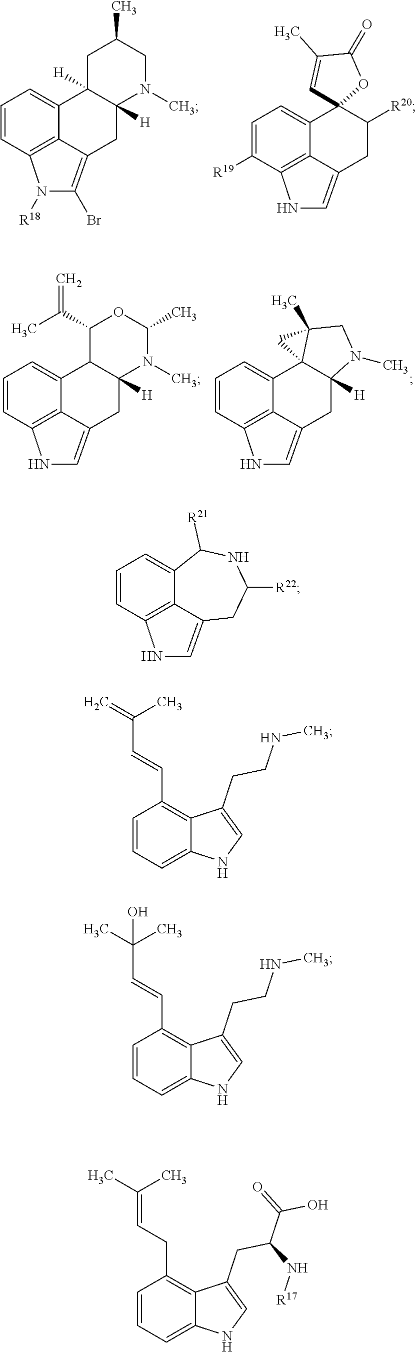

wherein R⁸ is selected from the group consisting of hydrogen and hydroxyl;

R⁹ is selected from the group consisting of α or β hydrogen, and α or β hydroxyl;

R¹⁰ is selected from the group consisting of hydrogen, hydroxyl,

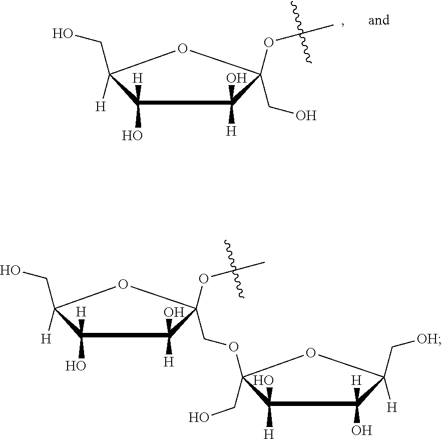

R¹¹ is selected from the group consisting of α and β hydrogen;

R¹² is selected from the group consisting of α or β hydrogen, α or β hydroxyl, and α or β acetoxy;

R¹³ is selected from the group consisting of α and β hydrogen;

R¹⁴ is selected from the group consisting of hydrogen and

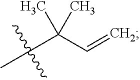 [SMILES: *C(C)(C)C=C]

R¹⁵ is selected from the group consisting of methyl, CH₂OH, COH,

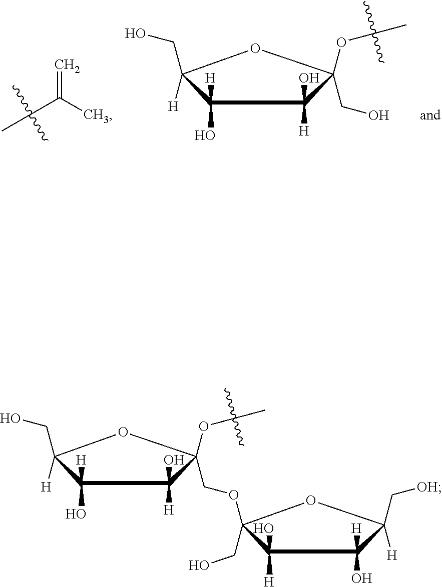

R¹⁶ is selected from the group consisting of methyl, CH₂OH, and hydroxyl;

R¹⁷ is selected from the group consisting of hydrogen and methyl;

R¹⁸ is selected from the group consisting of hydrogen and methoxy;

R¹⁹ is selected from the group consisting of hydrogen and chloride;

R²⁰ is selected from the group consisting of α and β NHCH₃;

R²¹ is selected from the group consisting of α and β

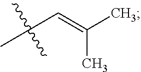 [SMILES: *C=C(C)C]

and

R²² is selected from the group consisting of α and β COOH.

Thus, in various embodiments, compounds of the present invention include:

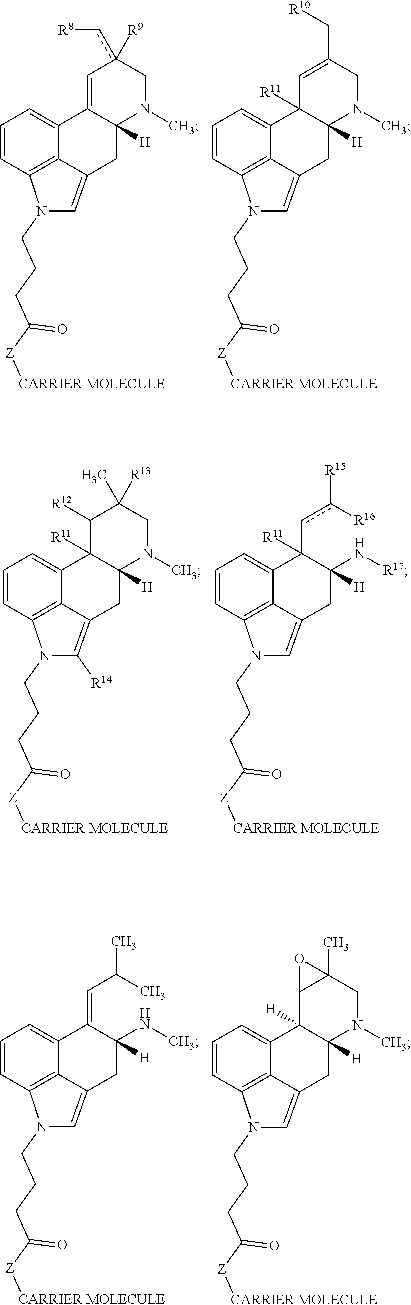

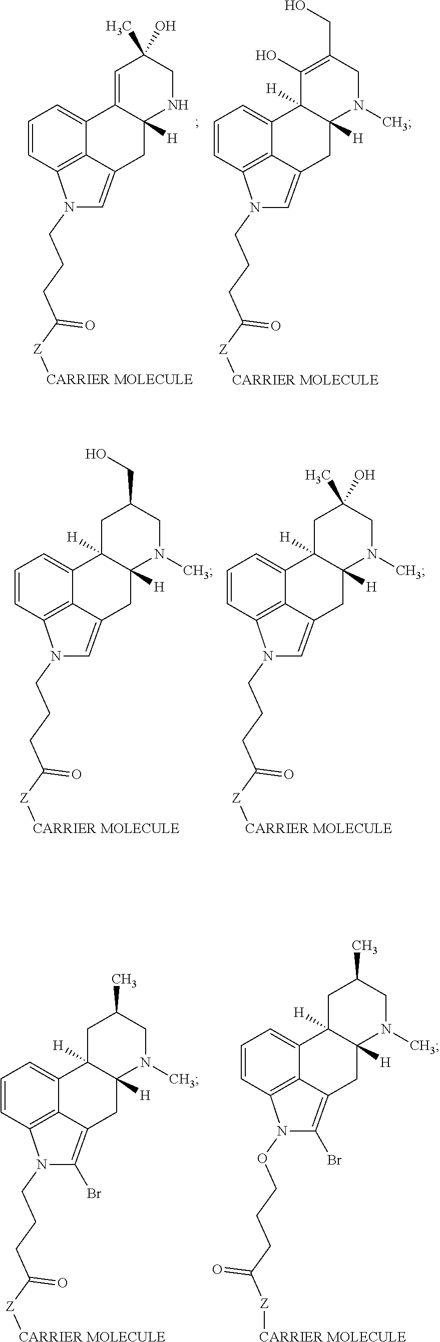

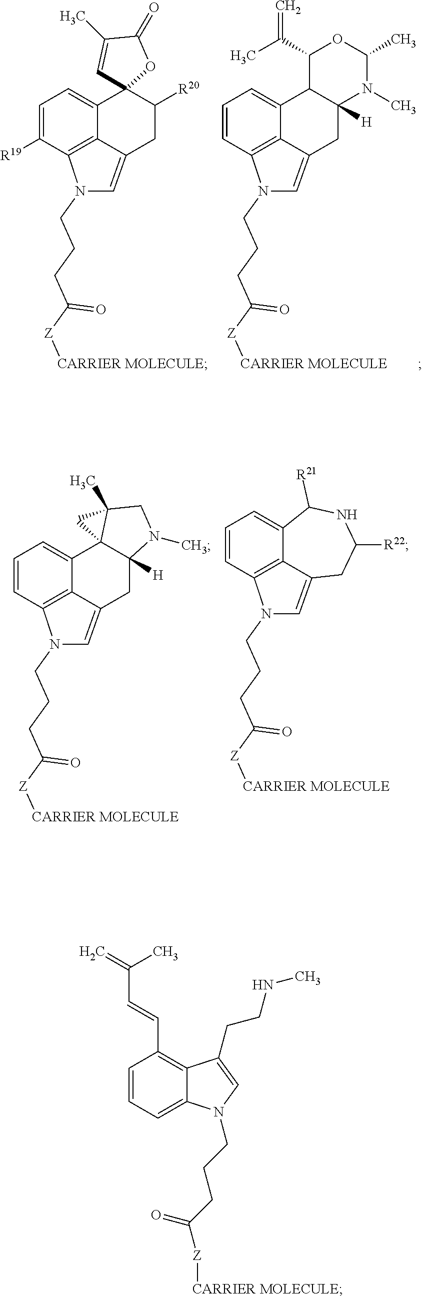

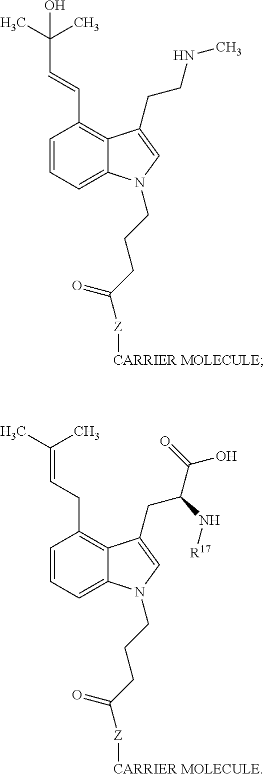

In the aforementioned disclosed compounds, the carrier molecule is not particularly limited and may include, but is not limited to, a peptide or a protein. Exemplary proteins include a suitable immunogenic protein, which may include human serum albumin, bovine serum albumin, chicken globulin, ovalbumin, keyhole limpet hemocyanin, polyarginine, polyhistidine, polytyrosine, polyserine, polyaspartate, and polylysine.

Also disclosed herein are methods for treatment of a subject or animal, such as a mammal exhibiting one or more physical manifestations of ergot-based toxicity. Various methods include administering a therapeutic or immunogenic amount of one or more of the aforementioned compounds and treating the one or more physical manifestation of ergot-based toxicity in the subject.

All of the MATERIALS and METHODS disclosed and claimed herein can be made and executed without undue experimentation in light of the present disclosure. While the compositions and methods have been described in terms of preferred embodiments, it is apparent to those of skill in the art that variations maybe applied to the MATERIALS and METHODS and in the steps or in the sequence of steps of the methods described herein without departing from the concept, spirit and scope herein. More specifically, certain agents that are both chemically and physiologically related may be substituted for the agents described herein while the same or similar results would be achieved. All such similar substitutes and modifications apparent to those skilled in the art are deemed to be within the spirit, scope and concept as defined by the appended claims.

1. A composition for treating ergot-based toxicity in a subject, the composition comprising one or more compounds selected from the group consisting of: 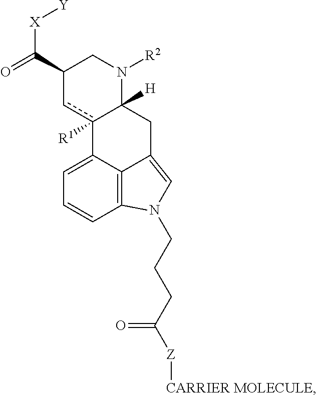
wherein
R¹ is selected from the group consisting of hydrogen and null, in the case of a double-bond between the C-3 and C-4 carbons;
R² is selected from the group consisting of a methyl and hydrogen;
X is selected from the group consisting of a bond, carbon, nitrogen, oxygen, an amine, an amide, an ester, and an ether;
Y is selected from the group consisting of hydrogen, methyl, ethyl, 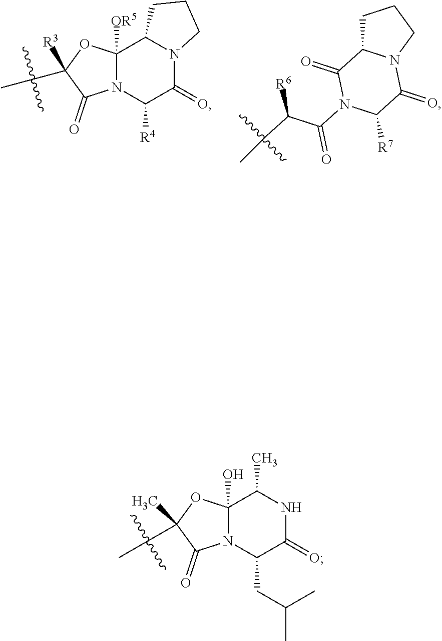
Z is selected from oxygen and nitrogen;
R³ is selected from the group consisting of methyl, ethyl, n-propyl, isopropyl, sec-butyl;
R⁴ is selected from the group consisting of benzyl, ethyl, isopropyl, isobutyl, sec-butyl, n-butyl, 2-methyl-n-butyl, 2-methyl-n-propyl, and ethyl(methyl)sulfane;
R⁵ is selected from the group consisting of hydrogen and methoxy;
R⁶ is selected from the group consisting of isopropyl and sec-butyl; and
R⁷ is selected from the group consisting of benzyl, ethyl, isopropyl, isobutyl, and sec-butyl.

2. The composition of claim 1, wherein the carrier molecule is selected from the group consisting of human serum albumin, bovine serum albumin, chicken globulin, ovalbumin, keyhole limpet hemocyanin, polyarginine, polyhistidine, polytyrosine, polyserine, polyaspartate, and polylysine.

3. The composition of claim 1, wherein at least one of the compounds has the following structure:

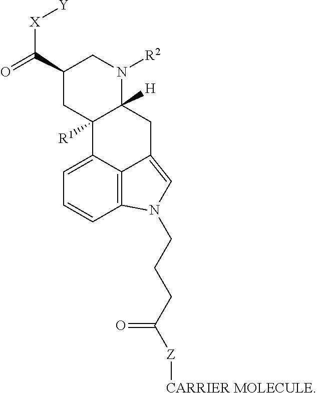

4. The composition of claim 1, wherein at least one of the compounds has the following structure:

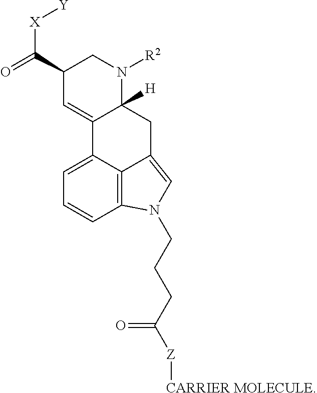

5. The composition of claim 1, wherein at least one of the compounds has the following structure:

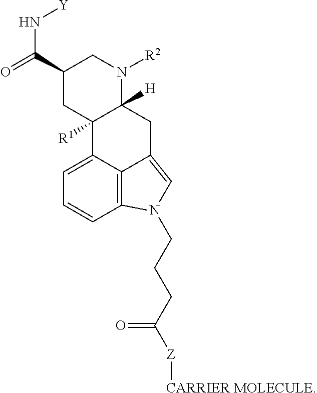

6. The composition of claim 1, wherein at least one of the compounds has the following structure:

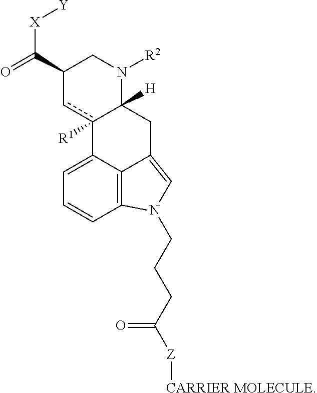

7. The composition of claim 1, wherein at least one of the compounds has hydrogen at R¹.

8. The composition of claim 1, wherein at least one of the compounds is selected from the group consisting of

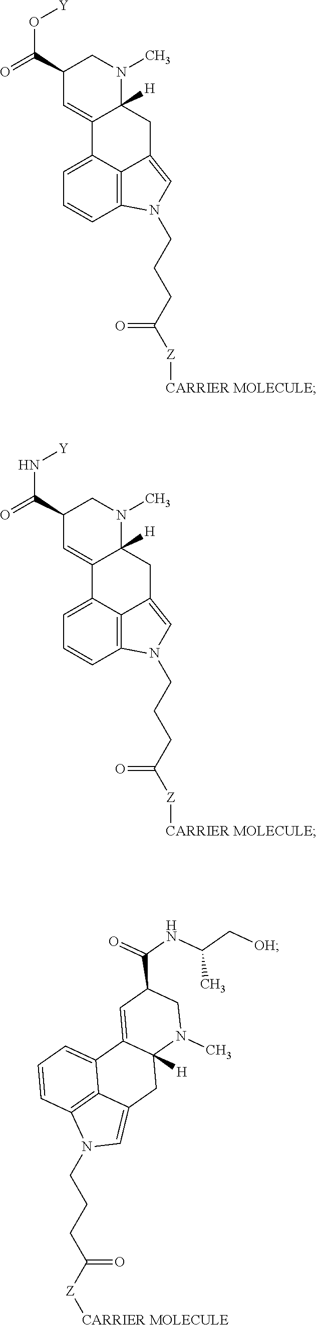

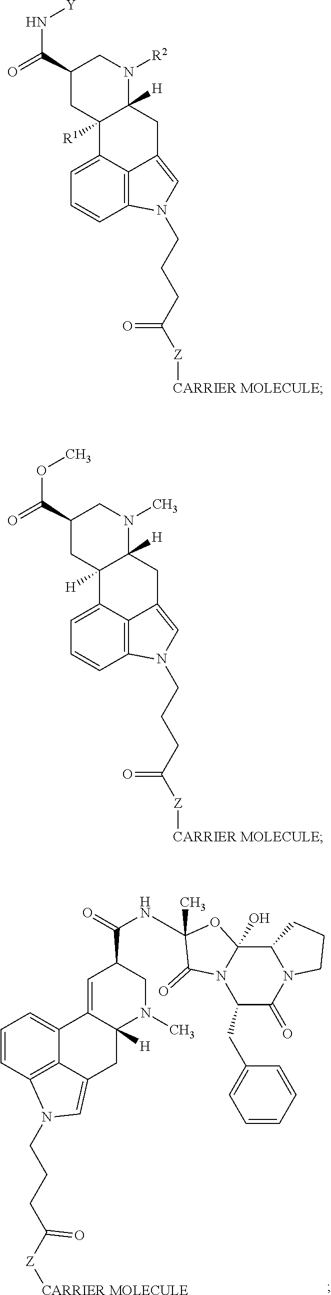

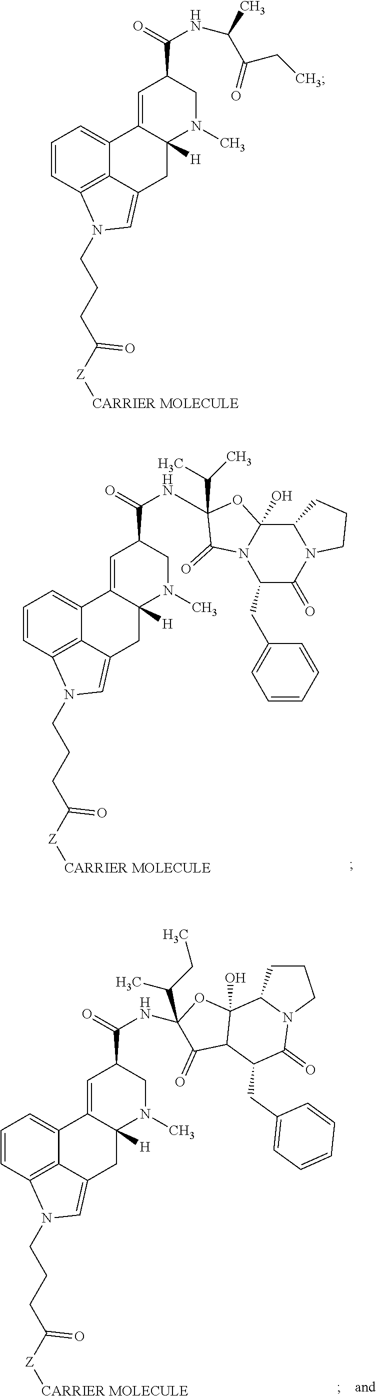

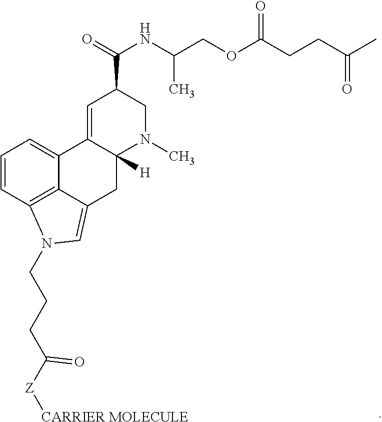

9. A composition for treating ergot-based toxicity in a subject, the composition comprising one or more clavines bonded to a carrier molecule.

10. The composition of claim 9, wherein the composition is selected from at least one of the group consisting of: 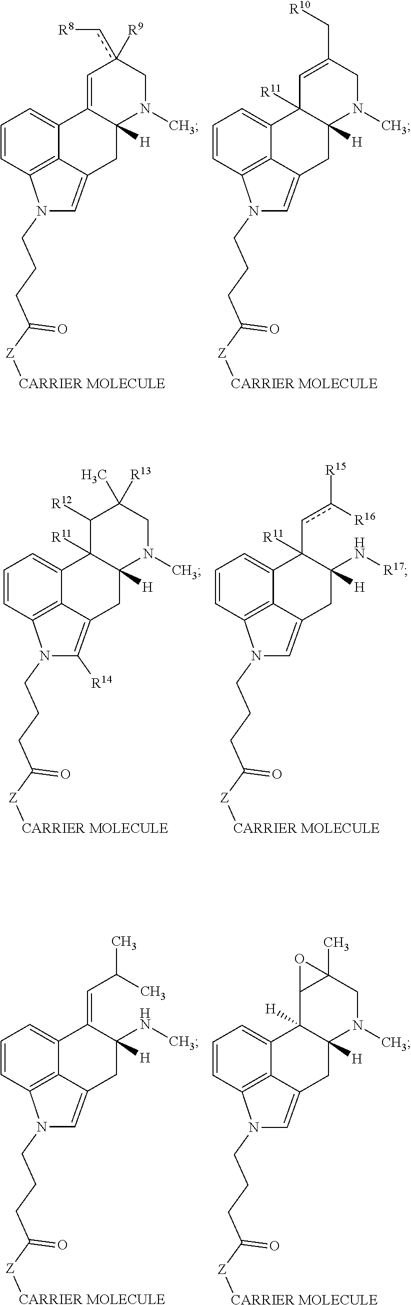 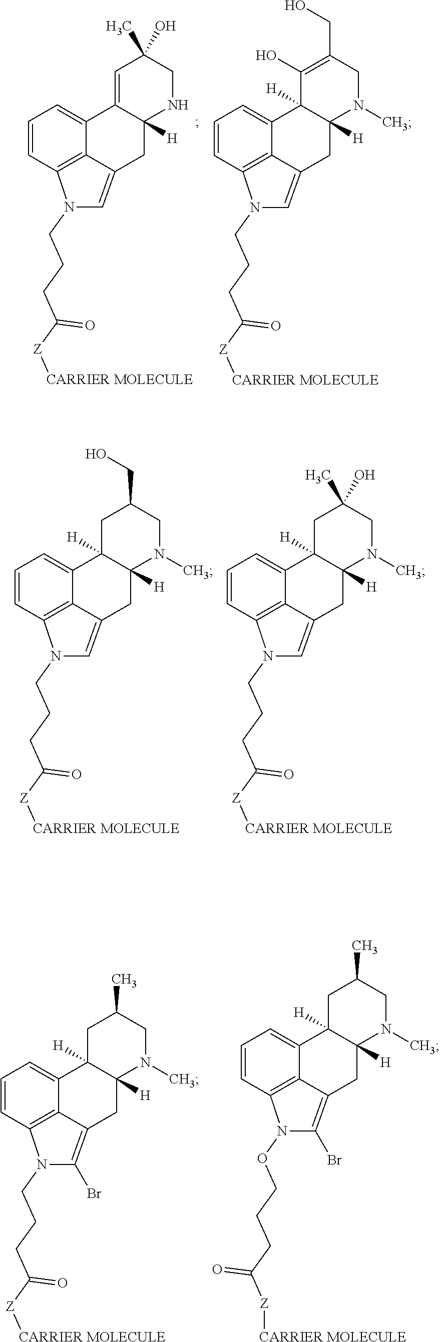 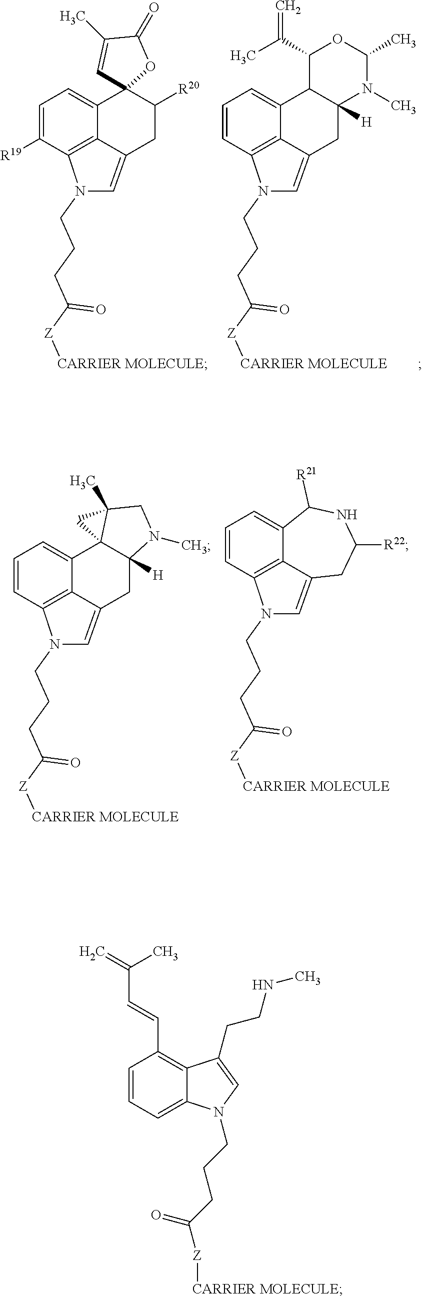 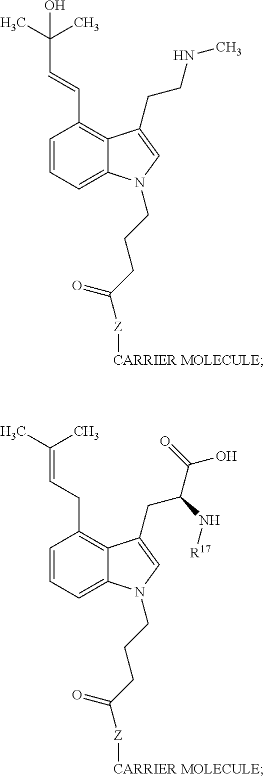
R⁸ is selected from the group consisting of hydrogen and hydroxyl;
R⁹ is selected from the group consisting of α or β hydrogen, and α or β hydroxyl;
R¹⁰ is selected from the group consisting of hydrogen, hydroxyl, 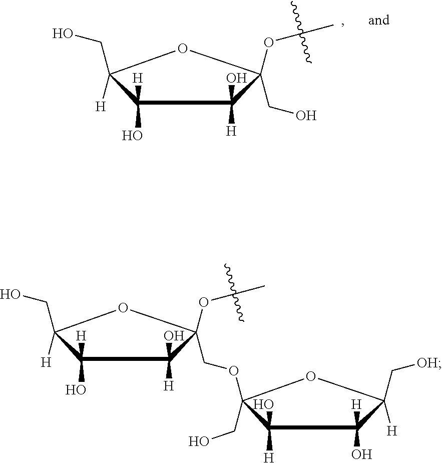
R¹¹ is selected from the group consisting of α and β hydrogen;
R¹² is selected from the group consisting of α or β hydrogen, α or β hydroxyl, and α or β acetoxy;
R¹³ is selected from the group consisting of α and β hydrogen;
R¹⁴ is selected from the group consisting of hydrogen and 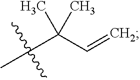 [SMILES: *C(C)(C)C=C]
R¹⁵ is selected from the group consisting of methyl, CH₂OH, COH, 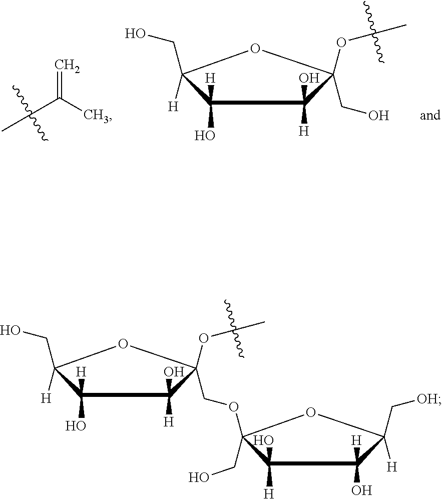
R¹⁶ is selected from the group consisting of methyl, CH₂OH, and hydroxyl;
R¹⁷ is selected from the group consisting of hydrogen and methyl;
R¹⁸ is selected from the group consisting of hydrogen and methoxy;
R¹⁹ is selected from the group consisting of hydrogen and chloride;
R²⁰ is selected from the group consisting of α and β NHCH₃;
R²¹ is selected from the group consisting of α and β 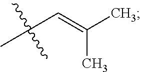 [SMILES: *C=C(C)C]
and
R²² is selected from the group consisting of α and β COOH.

11. A composition for preventing ergot toxicity, wherein at least one compound in the composition is: 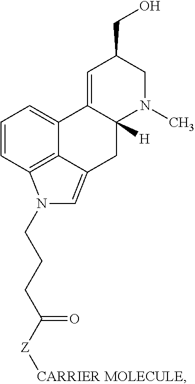
wherein Z is selected from oxygen and nitrogen.

12. A method for treating a subject exhibiting one or more physical manifestations of ergot-based toxicity, the method comprising administering a composition of claim 1.

13. A method for treating a subject exhibiting one or more physical manifestations of ergot-based toxicity, the method comprising administering a composition of claim 10.
